(19) United States
(12) Patent Application Publication
(10) Pub. No.: US 2026/0210943 A1
(43) Pub. Date: Jul. 23, 2026

(54) BRAWNIN AGONISTS FOR USE IN THE TREATMENT OF AXONAL METABOLIC DISORDERS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE CLAUDE BERNARD LYON 1, Villeurbanne (FR); ASSOCIATION FRANCAISE CONTRE LES MYOPATHIES (A.F.M.), Paris (FR)

(72) Inventors: Julien COURCHET, Lyon (FR); Sozerko YANDIEV, Lyon (FR)

(21) Appl. No.: 19/157,843

(22) PCT Filed: Mar. 8, 2024
(86) PCT No.: PCT/EP2024/056176

§ 371 Date: Aug. 19, 2025

(30) Foreign Application Priority Data
Mar. 9, 2023 (EP) 23305322.2

(51) Int. Cl. G01N 33/50; A61K 31/7105; C12N 5/0793; C12N 15/113; G01N 33/68

(52) U.S. Cl. CPC G01N 33/5058; A61K 31/7105; C12N 5/0619; C12N 15/113; G01N 33/6896; C12N 2310/11; C12N 2310/14; C12N 2503/02

(57) ABSTRACT

Using primary mouse neuronal cultures and mouse models, the present inventors have highlighted the role of a new effector, BRAWNIN, in neuronal metabolic balance and cortical axon branching. The present inventors have shown that BRAWNIN expression is necessary and sufficient for cortical axon branching. The present inventors have particularly demonstrated that BRAWNIN expression allows completely restoring impaired axon branching phenotypes in an impaired axon development mouse model. The present invention therefore pertains to a BRAWNIN agonist for use in the treatment of axonal metabolic disorders. The present invention further pertains to screening methods for the identification of novel therapeutic compounds based on BRAWNIN expression in a cellular model.

### TECHNICAL FIELD

The present invention pertains to novel therapeutic ways for treating axonal metabolic disorders. The present invention particularly pertains to BRAWNIN agonists for use in the treatment of axonal metabolic disorders.

### BACKGROUND ART

Axon morphogenesis is a multistep process culminating with the formation of axonal branches to form a network that is later refined through a process of selection of functional contacts and elimination by pruning (Lewis, Courchet & Polleux, The Journal of cell biology 202, 837-848 (2013); Kalil & Dent Nature Reviews Neuroscience 15, 7-18 (2014)). The formation and stabilization of axonal branches, collectively referred to as “axon branching”, relies on the activation of intracellular signaling pathways mediating a cascade of cellular events such as cytoskeleton remodeling, addition of membrane and local protein translation (Schwarz et al. The Journal of biological chemistry 270, 10990-10998 (1995); Dent & Kalil, The Journal of neuroscience: the official journal of the Society for Neuroscience 21, 9757-9769 (2001); Dent et al. The Journal of neuroscience: the official journal of the Society for Neuroscience 19, 8894-8908 (1999). Brosig et al., Cell reports 29, 2028-2040.e8 (2019); Spillane et al., The Journal of neuroscience: the official journal of the Society for Neuroscience 32, 17671-17689 (2012); Spillane, et al., Cell reports 5, 1564-1575 (2013)) that are highly taxing energetically and are thought to induce a local increase in the metabolic turnover. Evidence suggests that the rapid consumption of metabolic molecules such as ATP limits their spatial diffusion in the axon (Sun et al., Cell reports 4, 413-419 (2013); Matsumoto et al. Mol Cell Neurosci 119, 103704 (2022)), implying that cellular energy production must match the local demand.

A large body of work focused on the regulation of metabolic homeostasis in the brain, owing to the fundamental importance of the energy metabolism in synaptic function and neurodegeneration. Many, if not most, neurodegenerative diseases are associated to metabolic deregulation, and it is now well established that altered energy metabolism is one of the hallmarks of neurodegenerative diseases (David M. Wilson III et al., Cell 186, Feb. 16, 2023). The axon is especially sensitive to decrease in cellular energy due to its length and morphology, and many neurodegenerative diseases start with axon degeneration, such as for example diseases like Charcot-Marie-Tooth disease or Spinal Muscular Atrophy.

In contrast and until recently, fewer studies focused on the specificities of metabolic pathways in immature neurons. Altered mitochondrial metabolism is linked to increased risk of various neurodevelopmental disorders including Autism Spectrum Disorders (Chalkia et al., Jama Psychiat 74, 1161 (2017); Wang, et al., Plos Genet 12, e1006391 (2016)). Mitochondrial proteins are over-represented in protein interaction network of ASD risk genes (Murtaza et al. Neuron-specific protein network mapping of autism risk genes identifies shared biological mechanisms and disease-relevant pathologies. Cell Reports 41, 111678 (2022)). Mitochondrial defects can cause an increased ROS production, which has been linked to defects in cortical circuits formation (Fernandez et al. Neuron 102, 1127-1142.e3 (2019)).

Thus, it is now accepted that axonal metabolism plays a central role in neuronal development and circuits formation, as well as in the maintenance of the function and integrity of neural circuits and axonal projections. Yet the molecular mechanisms ensuring a local control of metabolic functions are still poorly understood. Previous studies have identified the implication of a Serine/Threonine kinase called NUAK1 (also known as AMPK-related protein kinase 5(ARK 5 )) in the control of cortical axon branching through the regulation of mitochondria capture at immature presynaptic sites (Courchet et al. Cell 153, 1510-1525 (2013)). NUAK1 mutations induce metabolic dysregulations leading to impaired axonal branching and altered development of neural circuits in a mouse model of Autism Spectrum Disorders (Courchet V et al, Nature communications 2018). More largely, mutations in NUAK1 causing axonal metabolic disruptions have been associated with several conditions including Autism Spectrum Disorders (ASD) (lossifov et al., Neuron 74, 285-299 (2012); lossifov et al., Nature 515, 216-221 (2014)), Attention Deficit/Hyperactivity Disorders (ADHD) (Alemany et al., American journal of medical genetics. Part B, Neuropsychiatric genetics: the official publication of the International Society of Psychiatric Genetics (2015) doi:10.1002/ajmg.b.32341), cognitive impairment (Johnson et al., Nature Neuroscience 19, 223-232 (2016)) or hydrocephaly (Vojinovic et al. Nature communications 9, 3945 (2018)).

However, the exact mechanisms and effectors involved in axonal metabolic regulation remain unknown. Further investigations are therefore essential to unravel the mechanisms by which NUAK1 and potential other effectors regulate axonal metabolism. The identification of new actors of the axonal metabolic pathway could allow identifying new therapeutic targets for neurological disorders associated with axonal metabolic impairment.

### SUMMARY

The invention is defined by the claims.

Using primary mouse neuronal cultures and in utero plasmid electroporation, the present inventors have demonstrated that the serine/threonine kinase NUAK1 promotes the development of axons through the regulation of axonal mitochondrial metabolism. The present inventors have identified that the expression and splicing of the gene BRAWNIN (also called UQCC6 or C12orf73), which codes the microprotein BRAWNIN involved in the formation of mitochondrial respiratory chain supercomplexes, is altered in the cortex of mice with a mutation in the gene NUAK1. More importantly, the present inventors have shown that BRAWNIN expression is necessary and sufficient for cortical axon branching. The present inventors have particularly demonstrated that BRAWNIN expression restores completely the impaired mitochondrial function and associated axon branching phenotypes in an impaired axon development mouse model.

These results open up new promising therapeutic ways for the treatment of various neurological disorders, in particular those associated with metabolic imbalance including axonal metabolic disorders. Metabolic activity in the axon is controlled by cellular mechanisms that participate to both development and maintenance of the axon, and that when perturbed lead to abnormal development (neurodevelopmental diseases) and abnormal maintenance (neurodegeneration).

Therefore, according to a first embodiment, the present invention pertains to a BRAWNIN agonist for use in the treatment of axonal metabolic disorders.

The present inventors have further shown that BRAWNIN is correlated with axon branching and that, accordingly, it could be used as an efficient marker for screening compounds having positive effects on axon branching.

Accordingly, the present invention also pertains to a method for screening candidate therapeutic compounds, wherein said method comprises:
a. Culturing a cellular model of an axonal metabolic disorder in presence of a candidate compound;
b. Assaying the effects of said candidate compound on the expression level of Brawnin in said cellular model; and
c. Selecting said compound based on said effects.

### BRIEF DESCRIPTION OF DRAWINGS

FIG. 1: Axonal metabolism impacts the growth and ramification of axons: Quantification of axon length and collateral branches of 5DIV neurons in the indicated conditions: (A-B) medium containing increasing concentrations of glucose or pyruvate (PYR). (C-D) 25 mM glucose-containing medium with increasing doses of rotenone.

FIG. 2: mitochondrial metabolism is altered in NUAK1-deficient neurons: (A-B) measurement of glucose metabolism in 5DIV cortical neurons cultures from the indicated genotypes. ExtraCellular Acidification Rate (ECAR) and Oxygen Consumption Rate (OCR) were normalized to protein dosage. (C) Citrate Synthase enzymatic activity relative to protein quantity (normalized to WT). Analysis: One-way ANOVA with Dunnett's multiple comparison test. (D) Measurement of the mitochondrial membrane potential (ΔΨm) in the axon of NUAK1^{F/F} neurons expressing a control (WT) or CRE-expressing plasmid (KO). Bars: average±95% CI. Analysis: Unpaired t-test with Welch's correction

FIG. 3: upregulation of axonal metabolism rescues axon branching in NUAK1-deficient neurons: (A) Quantification of axon collateral branches in the indicated conditions. Statistical tests: Kruskal-Wallis test with Dunn's post-test (each condition compared to untreated WT condition). (B) Effect of L-Car on the mitochondrial membrane potential (ΔΨm) of WT and KO neurons. Each point represents average values for a given neuron. Bars: average±95% CI. Analysis: Unpaired t-test with Welch's correction.

FIG. 4: Transcriptomic signature associated to Nuak1 knockout in cortical neurons: (A) Graph comparing Differentially Expressed Genes (log2FC ><0.25, p value<0,01) upregulated (red) and downregulated (blue) in vivo or in vitro following Nuak1 KO. (B-C) RT-qPCR validation of Brawnin mRNA downregulation within the cortex of E18.8 Nuak1 KO mice (B), as well as in primary neuronal cultures (C).

FIG. 5: Alterations of the splicing pattern of Brawnin in Nuak1 knockout neurons: (A) Schematic representation of the splicing donor sites of Brawnin transcript and corresponding splicing isoforms, and splicing profile in WT and NUAK1 KO cortices. (B-C) Quantifications of the abundance of the three splicing isoforms in WT and NUAK1 KO primary neurons (B) and cortices (C). Each form is represented as a percentage of total amount of Brawnin transcripts.

FIG. 6: BRAWNIN inactivation impairs axonal metabolism and disrupts axon development: (A) Quantification of axon collateral branches in the indicated conditions. Statistical tests: Mann-Whitney. (B) Effect of Brawnin shRNA on the mitochondrial membrane potential (ΔΨm). Each point represents average values for a given neuron.

FIG. 7: Metabolic consequences of an inhibition of BRAWNIN expression in mouse cortical neurons. (A) Effect of Brawnin shRNA on the mitochondrial membrane potential (ΔΨm). Each point represents average values for a given neuron. Data: average±SEM. Statistical test: Two-tailed Mann-Whitney. (B) Mitochondrial respiration evaluated by Seahorse assay in BRAWNIN knocked-out neurons. The Oxygen Consumption Rate (OCR) was measured in cortical neurons cultured for 5 days in vitro and transduced with shRNA against Brawnin. OCR values are normalised to cell number. Basal and maximal respiration values are represented in (C) and (D) respectively. Data: average±SEM. Statistical test: Two-tailed Mann-Whitney.

FIG. 8: Re-expression of BRAWNIN increases axonal metabolism and is sufficient to restore axonal branching development: (A) Axon collateral branches of 5DIV neurons in the indicated conditions. Statistical tests: Kruskal-Wallis test with Dunn's post-test. (B) Measurements of the mitochondrial membrane potential (ΔΨm) in the indicated conditions. Bars: average±95% CI. Statistical tests: Kruskal-Wallis test with Dunn's post-test (each condition compared to the control condition).

### DETAILED DESCRIPTION OF THE INVENTION

The present inventors have highlighted the role of a novel effector, BRAWNIN, in mitochondrial metabolic activity in the axon. The results presented herein show that BRAWNIN expression is necessary for mitochondrial metabolism and collateral branch stabilization in developing cortical neurons. The present inventors have further shown that it is possible to restore impaired axon branching phenotypes by overexpressing BRAWNIN in an impaired axon development mouse model.

Therefore, the present invention pertains to targeting BRAWNIN for the treatment of disorders associated with mitochondrial dysfunction and axonal metabolic imbalance.

According to a first aspect, the present invention therefore pertains to a BRAWNIN agonist for use in the treatment of axonal metabolic disorders.

A method for the treatment of axonal metabolic disorders comprising a step of administering a therapeutically effective amount of a BRAWNIN agonist to a patient in need thereof is also disclosed therein.

“BRAWNIN”, also called “UQCC6” or “C12orf73”, is a microprotein that has been shown to be involved in mitochondrial respiratory chain assembly (Pathak D et al., The Journal of biological chemistry 290, 22325-22336(2015); Tao et al., Developmental neurobiology 74, 557-573 (2014)). It is a 71-amino acid residues protein that is encoded by the BRAWNIN gene. The amino acid sequence of BRAWNIN is e.g. available under reference Q69YU5 in the Uniprot database.

According to the present invention, a BRAWNIN “agonist” refers to a natural or synthetic compound capable of significantly increasing the intracellular level and/or activity of BRAWNIN in neurons, in particular in vivo. The skilled person knows how to determine whether a compound qualifies as a “BRAWNIN agonist” according to the present invention. Knowing the amino-acid sequence of the BRAWNIN protein, and the nucleotide sequence of the corresponding gene, the skilled person can easily determine the expression level of BRAWNIN by using several techniques that are routine to the skilled person.

The expression level of the BRAWNIN protein can be evaluated by measuring the expression level of the BRAWNIN gene. The expression level of a gene is typically determined by analyzing the mRNA(s) transcribed from that gene. These nucleic acid molecules can typically be extracted from a sample and analyzed by standard methods. One method commonly used is to subject the isolated mRNAs to reverse transcription (“RT”) coupled with polymerase chain reaction (“PCR”) amplifications using oligonucleotide primers specific to the genes of interest. Quantification of mRNAs can typically be performed using one of two real-time quantification technologies called SYBR Green® or TaqMan®.

The expression level of BRAWNIN can also be directly evaluated by measuring the amount of the BRAWNIN protein detected in the test sample. Such methods typically involve contacting the sample to be analyzed with an agent capable of specifically binding the target protein. This agent is usually a polyclonal or monoclonal antibody. The presence of the protein is then typically detected by standard immunodetection methods after separation of the proteins by electrophoresis (technique also called “Western blotting”) or by immunoassays by direct, indirect, competition or immunocapture methods (techniques also called “ELISA”). The formation of a complex between the protein of interest and the antibody(s) targeting said protein is usually detected and quantified by measuring an enzymatic reaction generating a colored, chemiluminescent or fluorescent product.

In the context of the present invention, the BRAWNIN agonist can e.g. be the BRAWNIN protein per se. As mentioned above, BRAWNIN is a small protein that can be easily synthetized and administered to a subject. In this context, the BRAWNIN agonist refers to the BRAWNIN polypeptide. The BRAWNIN agonist can also refer to a functional fragment of the BRAWNIN protein, i.e. to a BRAWNIN fragment that has the same activity as the mature BRAWNIN protein. Such a functional fragment is typically capable, upon administration, of restoring axonal branching in an impaired axon development mouse model as achieved by the mature BRAWNIN protein.

The skilled person is familiar with formulation techniques for administering a known protein to a subject and will therefore know the best excipients and route of administration to be used.

The BRAWNIN agonist used in the context of the present invention can also be a BRAWNIN-encoding polynucleotide. According to this embodiment, the treatment used for treating the axonal metabolic disorder is therefore a gene therapy.

Gene therapy may be carried out by means of supplementation of target cells with a functional BRAWNIN. Production of a suitable gene product may be achieved by using recombinant techniques. For example, a suitable vector may be inserted into a host cell and expressed in that cell. Gene therapy is typically achieved by administering a polynucleotide encoding the target gene to the patient.

Typically, said polynucleotide is comprised in an expression cassette.

An “expression cassette” refers to a linear or circular nucleic acid molecule. This expression cassette also refers to DNA and RNA sequences which allow for the production of a functional nucleotide sequence in a suitable host cell. Typically, the expression cassette comprises a polynucleotide encoding BRAWNIN operatively linked to at least one transcriptional regulatory sequence. Typically, the expression cassette comprises a polynucleotide encoding the BRAWNIN protein, said polynucleotide being operatively linked to at least one transcriptional regulatory sequence for the expression of BRAWNIN protein in target cells.

The expression cassette can also include sequences required for proper translation of the nucleotide sequence of interest.

The expression cassette may additionally contain selection marker genes. Typically, the cassette comprises in the 5′-3′direction of transcription, a transcriptional and translation initiation region, a polynucleotide encoding the BRAWNIN protein, a transcription and translation termination region functional in mammalian cells.

The expression cassette may also include a multiple cloning site. In addition to the components mentioned above, the expression cassette of the present invention may comprise the components required for homologous recombination.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the term “transcriptional regulatory sequence”, “transcription regulatory sequence” or “regulatory sequences” refers to nucleotide sequences influencing the transcription, RNA processing or stability, or translation of the associated (or functionally linked) nucleotide sequence to be transcribed. The transcriptional regulatory sequence may have various localizations with the respect to the nucleotide sequences to be transcribed. The transcriptional regulatory sequence may be located upstream (5′ non-coding sequences), within, or downstream (3′non-coding sequences) of the sequence to be transcribed (e.g., polynucleotide encoding the BRAWNIN protein). The transcription regulating nucleotide sequences may be selected from the group consisting of enhancers, promoters, translation leader sequences, introns, 5′-untranslated sequences (5′UTR), 3′-untranslated sequences (3′UTR), and polyadenylation signal sequences. They include natural and synthetic sequences as well as sequences, which may be a combination of synthetic and natural sequences. As is noted above, the term “transcriptional regulatory sequence” is not limited to promoters. However, transcriptional regulatory sequence of the invention may comprise at least one promoter sequence (e.g., a sequence localized upstream of the transcription start of a gene capable to induce transcription of the downstream sequences), and/or at least one 3′UTR and/or one 5′UTR.

The expression cassette is typically comprised in an expression vector.

The term “vector” refers to a nucleic acid sequence capable of transporting into a cell another nucleic acid to which the vector sequence has been linked. The term “expression vector” includes any vector containing a gene construct or an expression cassette in a form suitable for expression by a cell. The “expression vector” may be any recombinant vector capable of expression of a BRAWNIN protein or fragment thereof. More particularly, the expression vectors used can be derived from bacterial plasmids, transposons, yeast episomes, from insertion elements, from yeast chromosomal elements, from viruses such as an adeno-associated virus (AAV) vector, a lentivirus vector, a retrovirus vector, a replication competent adenovirus vector, a replication deficient adenovirus vector and a gutless adenovirus vector, a herpes virus vector, baculoviruses, blinked as SV40 virus, the vaccinia virus, fox pox viruses, pseudorabies viruses.

AAV and lentivirus vectors have emerged as the vectors of choice for gene transfer to the central nervous system as they mediate efficient long-term gene expression with no apparent toxicity. The expression cassette can be inserted into the expression vector by methods well known in the art.

The BRAWNIN-encoding polynucleotide may be introduced into a target cell by means of any procedure known for the delivery of nucleic acids to the nucleus of cells, ex vivo, on cells in culture or removed from an animal or a patient, or in vivo. Such techniques e.g. include transfection, electroporation, microinjection, transduction, cell fusion, DEAE dextran, lipofection. Alternatively, one of the simplest and the safest way to deliver the BRAWNIN-encoding polynucleotide across cell membranes in vivo may involve the direct application of high concentration free or naked polynucleotides (typically mRNA or DNA).

According to another embodiment, the BRAWNIN agonist used in the context of the present invention can be a nucleotide sequence allowing for the expression of the BRAWNIN gene.

The present inventors have indeed shown that dysfunction in BRAWNIN expression such as that observed in NUAK1-mutated cells results from an altered differential splicing of BRAWNIN in its 5′UTR region. Therefore, a promising way for restoring BRAWNIN expression is to correct this impaired splicing. This can be achieved by using nucleotide constructs such as oligonucleotide constructs (including antisense RNA molecules such as morpholinos or antisense oligomers) or siRNAs so as to prevent the incorrect splicing of the BRAWNIN gene.

As an example, a potential strategy would be the development of morpholinos or antisense oligomers-based exon skipping targeting some of the alternatively spliced isoforms of BRAWNIN mRNA, in order to decrease the form being produced in pathological conditions and favor the form being produced in non-pathological conditions. Alternative mechanisms can be proposed, including the modulation of RNA stability targeting specifically some alternatively spliced forms of BRAWNIN mRNA in a mechanism known as RNA interference.

All these constructs can be prepared by known methods. These include techniques for chemical synthesis such as, e.g., by solid phase phosphoramadite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides used in the context of the present invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone. Antisense oligonucleotides and siRNAs may be delivered in vivo alone or in association with a vector.

The BRAWNIN agonist used in the context of the present invention is preferably selected from the group consisting of the BRAWNIN protein or a functional fragment thereof, a BRAWNIN-encoding polynucleotide and a nucleotide sequence allowing for the expression of the BRAWNIN gene.

In the context of the present invention an “axonal metabolic disorder” refers to axonal disorders associated with a metabolic defect. A growing body of evidence support that metabolic regulation and especially mitochondrial metabolism is involved in the formation of the axon and of neural connections, in the central nervous system and also in the peripheral nervous system (Spillane et al., Cell reports 5, 1564-1575 (2013); Wong et al., Neuron 95, 852-868.e8 (2017); Courchet et al. Cell 153, 1510-1525 (2013); Cioni et al., Cell 176, 56-72.e15 (2019)). Neuronal metabolism has long been studied and it is acknowledged that the axon has metabolic specificities, such as a reliance on coupling with glial cells (oligodendrocytes and astrocytes) for metabolic support (Saab, Tzvetanova & Nave, Current opinion in neurobiology 23.6 (2013): 1065-1072). As early as 1979, it has been postulated that bioenergetic failure in the axon is associated with neuropathy and axon degeneration (Spencer et al., Annals of Neurology: Official Journal of the American Neurological Association and the Child Neurology Society 5.6 (1979): 501-507). This hypothesis has been largely demonstrated since and the link between metabolism and axon degeneration is now established (see for example Sasaki, Neuroscience research 139 (2019): 9-20”).

Axons are especially sensitive to alterations of metabolism and mitochondrial function because they constitute the vast majority (in some case 99 percent) of the cell size, because they are extremely elongated and have a cell/surface ratio extremely unfavorable. Thus, for the class of neurodegenerative diseases which are primarily caused by a loss of axonal projections, one early observation is an alteration of metabolic functions. Deficit in axonal energy metabolism are associated to various conditions, to cite a few: Multiple Sclerosis (Yi-Heng et al., Nature Metabolism 5.8 (2023): 1364-1381); Cambron et al. Journal of Cerebral Blood Flow & Metabolism 32.3 (2012): 413-424; López-Muguruza & Matute. International Journal of Molecular Sciences 24.16 (2023): 12912), axon degeneration/regeneration (Li et al., “Glial metabolic rewiring promotes axon regeneration and functional recovery in the central nervous system.” Cell metabolism 32.5 (2020): 767-785; Han et al., Cell metabolism 31.3 (2020): 623-641; Looser et al., Nature neuroscience (2024): 1-16), neurodegeneration (Beirowski, Neurobiology of Disease 170 (2022): 105751; Yang, Park & Lu, Molecular neurodegeneration 18.1 (2023): 49). Metabolic defects are now considered one of the hallmarks of neurodegenerative disorders (Wilson et al., Cell 186.4(2023 ): 693-714), and altered axonal metabolism is well studied in many pathological conditions.

Finally, the molecular mechanisms supporting axon development and axon maintenance/survival are often shared, and involve metabolic regulation (Oruganty-Das et al., Cell Metabolism 16, 789-800 (2012); Gunnewiek et al. Cell reports 31, 107538 (2020); Lewis et al., Nature communications 9, 5008 (2018); Fernandez et al., Neuron 102, 1127-1142.e3 (2019)). As an example, the kinase WNK1/2 play an evolutionarily conserved role in the development of the axon, by blocking the so-called Wallerian degeneration mechanism, which leads to axon degeneration in response to decrease in metabolic activity in the axon (decreased NAD and ATP levels—Izadifar et al., Neuron 2021 Volume 109, Pages 2864-2883.e8). Mutations in the genes WNK1/2 lead to peripheral neuropathies associated with axon degeneration. Hence, metabolic activity in the axon is controlled by cellular mechanisms that participate to both development and maintenance of the axon, and that when perturbed lead to abnormal development (neurodevelopmental diseases) and abnormal maintenance (neurodegeneration). This argument defines a group of diseases characterized by “axonal metabolic disorders”.

Axonal metabolic disorders are a well-defined group of disorders which can affect axonal formation or axon maintenance, and thus can manifest as neurodevelopmental or neurodegenerative diseases. Axonal metabolic disorders can be associated with extra-axonal phenotypes which are often secondary to axonal deficits, such as neuronal death. Axonal metabolic disorders affect neurons from the central nervous system, including cortical, hippocampal or cerebellar neurons, and can also affect the spinal cord in the case of diseases affecting the motor and sensory neurons. The skilled person knows which disorders qualify as an axonal metabolic disorder.

According to a specific embodiment, the “axonal metabolic disorder” treated in the context of the present invention can be selected from the group of conditions characterized by deficits on axonal metabolism. This group includes neurodevelopmental disorders including Autism Spectrum Disorders (ASD). This group also includes disorders characterized by a degenerescence of the axon and neuronal death, either isolated or associated to abnormal development. This group includes diseases of the central and motor nervous system such as Charcot-Marie-Tooth disease, mitochondrial ataxias, Spinal Muscular Atrophy (SMA).

“Autism Spectrum Disorders” or “ASD” a neurological and developmental disorder that affects patient's social interaction, communication, learning abilities and behavior. According to the Diagnostic and Statistical Manual of Mental Disorders (DSM-5) (American Psychiatric Association. (2013), people with ASD often have social communication deficits as well as restricted interests and repetitive behaviors that affect their ability to function in school, work, and other areas of life.

“Charcot-Marie-Tooth disease” or “CMT” is the commonest inherited neuromuscular disorder. It is a disease of the peripheral nerve (neuropathy) that results in decreased muscle strength and sensation (sensory-motor). It affects at least 1 in 2,500. The onset of clinical symptoms is in the first or second decade of life. Weakness starts distally in the feet and progresses proximally in an ascending pattern (Szigeti, Kinga, and James R. Lupski. European Journal of Human Genetics 17.6 (2009): 703-710). Management of CMT mainly involves physiotherapy and physiological activity so as to slow the disease's progression. Candidate drugs are currently being tested (such as PXT3003—Attarian et al. Orphanet Journal of Rare Diseases 16.1 (2021): 1-12; or IFB-088—Bai et al., Mol Neurobiol. 2022 Jul; 59(7):4159-4178) but not treatment has yet been approved for CMT.

“Ataxias” refers to a group of neurological disorders which affect coordination, balance and speech. They usually result from a damage in the cerebellum. Ataxias may cause difficulty with walking and balance, hand coordination, speech and swallowing, and eye movements. Ataxia is one of the most frequent symptoms of mitochondrial disease. “Mitochondrial ataxias” refer to a group of ataxias associated with a mitochondrial disease. They can be caused by disturbance of the cerebellum and its connections, involvement of proprioception or a combination of both (for review see Vernon, Hilary J., and Laurence A. Bindoff.,

Handbook of Clinical Neurology 155 (2018): 129-141 or Lopriore et al.,

Neurol Int. 2022 April 2; 14(2): 337-356).

“Spinal Muscular Atrophy” or “SMA” refers to a group of genetic disorders characterized by degeneration of anterior horn cells resulting in muscle atrophy and weakness. The most common SMA, accounting for over 95% of cases, is an autosomal recessive disorder that results from a homozygous deletion or mutation in the 5q13 survival of motor neuron (SMN1) gene (Kolb et al.,

Neurologic clinics 33.4 (2015): 831-846). SMA is classified under 4 subtypes depending on the age at which it develops. There is no cure approved for SMA. However, management of the disease includes exercises and equipment to help with movement and basic needs (including breathing and eating) as well as surgeries for treating spinal damages.

In the context of the present invention, the “patient” or “subject” is a mammal (e.g. a dog, a cat, a pig, a rodent or a primate). In a particular embodiment, the patient is a human.

In the context of the invention, the term “treating” or “treatment”, means reversing, alleviating, inhibiting the progress of the disorder or condition to which such term applies, or one or more symptoms of such disorder or condition. A “therapeutically effective amount” is intended for a minimal amount of active agent (e.g., BRAWNIN agonist) which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with a disorder.

A further aspect of the present invention pertains to a method for screening candidate therapeutic compounds, wherein said method comprises:
a. Culturing a cellular model of neural cells in presence of a candidate compound;
b. Assaying the effects of said candidate compound on the expression level of Brawnin in said cellular model; and
c. Selecting said compound based on said effects

Indeed, as explained above, the present inventors have shown that BRAWNIN is correlated with efficient axon branching. Accordingly, BRAWNIN expression can be used as an efficient marker for screening compounds having positive effects on axon branching.

The “cellular model” used according to this embodiment consists in a neural cell culture. According to a specific embodiment, said neural cell culture can be derived from iPSCs. Established culture methods can lead to the differentiation of iPSCs into various neuronal cell types pertinent for the study of axonal metabolic disorder, e.g. glutamatergic neurons (cortical neurons), motor neurons, or sensory neurons. The neural cell culture used in the context of the present invention can also be a primary neural cell culture, particularly a glutamatergic cortical neural cell culture, cerebellar neural cell culture, or spinal cord or dorsal root ganglia derived neural cell cultures. The cells may or may have not be treated so as to impair BRAWNIN expression, e.g. by inactivating NUAK1 expression. The cellular model is preferably a model of an axonal metabolic disorder.

As used herein, the term “induced pluripotent stem cells” or “iPS cells” or “iPSCs” refers to a type of pluripotent stem cell artificially derived from a non-pluripotent cell (e.g. a somatic cell). Induced pluripotent stem cells are identical to embryonic stem cells in the ability to form any adult cell, but are not derived from an embryo. For review on protocols allowing for the production of iPSCs, one can consult Shi, Yanhong, et al. (Nature reviews Drug discovery 16.2 (2017): 115-130). According to an embodiment of the invention iPS cells may be selected from any iPS cell line.

Typically, the “effect” as used herein, can refer either to a desired (for example, stimulation of BRAWNIN expression) or an undesirable effect (such as neurotoxicity) of a compound which is being screened for a therapeutic purpose. The skilled person in the art will understand what type of biological effect is to be assayed, according to the specific goal of the screening method. In the context of the present invention, the candidate compound is preferably selected when it allows for an increase (i.e. a statistically significant increase) of BRAWNIN expression.

The invention will now be further illustrated by means of the following examples. These examples are provided for illustrative purposes only and cannot be interpreted as limiting the scope of the invention.

### EXAMPLES

### Materials and Methods

### Animals

Mice breeding and handling was performed by following National Institutes of Health guidelines and the French and European legislation. Experimental protocols were approved by the CECCAPP Ethics committee (C2EA 15) of the University of Lyon. Time-pregnant females were maintained in a 12 hour light/dark cycle and obtained by overnight breeding with males of the same strain. Noon following breeding was considered as E0.5. Floxed NUAK1 mice (Nuak1^{tm1a(KOMP)Wtsi}) have been described previously (Courchet et al. Nature communications 9, 4289 (2018)). The NexCRE mouse line (Neurod6^{tm1(cre)Kan}) (Goebbels et al. Genesis (New York, N.Y.: 2000) 44, 611-621 (2006)) were provided by Sandrine Humbert (Grenoble Institute of Neuroscience, France). Animals were maintained on a C57BI/6J background.

### Primary Neuronal Culture and Ex Vivo Cortical Electroporation

The electroporation of dorsal telencephalic progenitors was performed by injecting plasmid DNA (1-2 μg/μL of endotoxin-free plasmid DNA) plus 0.5% Fast Green (Sigma; 1:20 ratio) using a Picospritzer III microinjector (Harvard Apparatus) into the lateral ventricles of isolated E15.5 embryonic mouse heads. Electroporations were performed with gold-coated electrodes (GenePads 5 mm, BTX) using an ECM 830electroporator (BTX) and the following parameters: five pulses of 100 milliseconds (msec), 150 msec interval, at 20 V. Immediately after electroporation, cortices were dissected in Hank's buffered salt solution (HBSS) supplemented with HEPES (pH 7.4; 2.5 mM), CaCl₂ (1 mM, Sigma), MgSO₄ (1 mM, Sigma), NaHCO₃ (4 mM, Sigma), and D-glucose (30 mM, Sigma), hereafter referred to as complete HBSS (cHBSS). Isolated cortices were dissociated in cHBSS containing papain (Worthington, 20 U/mg at least) for 20 minutes at 37° C. Cortices were washed once in cHBSS containing DNase I (2.5 mg/mL, Sigma), then 3 times in cHBSS before being dissociated. Cells were then plated at 125. 10³ cells per 35 mm glass bottom dish (MatTek) coated with poly-D-lysine (0.1 mg/mL) and laminin (0.01 mg/mL) and cultured for 5-7 days in Neurobasal medium supplemented with B27 (1×), N2 (1×), Glutamax (2 mM), and penicillin (10 U/mL)-streptomycin (0.1 mg/mL).

### In Utero Cortical Electroporation

Utero Cortical Electroporation were performed at E15.5 as described in Meyer-Dilhet & Courchet (STAR protocols 1, 100027 (2020)). A mix containing 1 μg/ul endotoxin-free plasmid DNA plus 0.5% Fast Green (Sigma; 1:20 ratio) was injected into one lateral hemisphere. Electroporation was performed using an ECM 830 electroporator (BTX) using four pulses of 45V with 500 msec interval to target cortical progenitors. Animals were sacrificed at Postnatal day 21 by terminal perfusion of 4% paraformaldehyde (PFA, Electron Microscopy Sciences) followed by 2 h post-fixation in 4% PFA.

For the L-Carnitine supplementation experiment, pregnant mice were treated with veterinary-approved L-Carnitine (Isulik 20, Sogeval laboratories) in the drinking water since detection of gestation (E13.5). Drug treatment was continued after birth and during lactation, and pups were sacrificed at weaning (P21) when cortical layer ⅔ PNs axons display complete branching patterns.

shRNAs

shRNAs against mouse BRAWNIN (shRNA1: TRCN0000269184; shRNA2: TRCN0000269131) were selected from The RNAi Consortium shRNA Library (TRC) from the Broad Institute. The pLKO.1 TRC-cloning vector vas digested with Agel-HF and EcoRI-HF, then purified with the NucleoSpin Gel and PCR clean up kit (Macherey-Nagel 740609.50). shRNA primers were annealed in NEBuffer 2 (NEB) and then ligated into the linearized pLKO.1 in rCutSmart buffer (NEB).

### Retro-Transcription and PCR

RNA from Nuak1 KO cortical neurons and cortices was extracted using Trizol Reagent (Ambion by Life Technologies) and the PureLink RNA MiniKit 12183018a (Invitrogen). cDNA synthesis was done using 500ng of RNA with SuperScript II (Invitrogen) and Oligo(dT) 12-18 (Invitrogen) primers. Splicing PCR reaction was done using Dream Taq DNA polymerase (ThermoScienfic) and C1000 Touch ThermoCycler (BioRad). Following the splicing PCR, each band corresponding to an isoform of BRAWNIN was isolated using UV transilluminator (FLX- 20M) and purified with NucleoSpin Gel and PCR clean up (Macherey-Nagel 740609.50). Obtained DNA was then sequenced via Sanger method by EurofinGenomics. qPCR reaction was performed using FastStart Universal SYBR Green Master Mix—ROX (Roche) and CFX Machine Connect Optics Module (BioRad).

### Metabolomic Flux Analyzes

We used a Seahorse XFe24 analyzer (Agilent) with the Glycolysis and Mito Stress test kits. Neurons were plated at 100,000 cells per well in Seahorse compatible 24 well plates and metabolic activity was measured at 7 days in vitro. The assay was conducted in Modified HBSS (1XHBSS supplemented with glucose (3.5g/L), Sodium pyruvate 1 mM, Glutamax 1X, CaCl2 1 mM, MgSO4 1 mM, pH 7.4). Following measurement, neurons were lysed in ice-cold lysis buffer (LB) and protein concentration was measured by Bradford for post-hoc normalization.

### Biochemical Assay

Biochemical assays were performed on mouse cortex samples from NUAK1 KO or NUAK1 WT mice. Samples were subjected to a chemical lysis using RIPA buffer (150 mM NaCl, 0,5 M EDTA ph8, 50 mM Tris pH 8; 1% NP-40, 0.5% sodium deoxycolate, 0.1% SDS) and followed by a mechanical lysis using the Precellys Evolution. Citrate Synthase enzymatic activity (EA) was determined by measuring absorbance using a PowerWave XS plate reader (BioTek). We used the Beer Lambert law EA=Mean V(ε×I) where I is the width of the tank (I=0.4 cm), ε=13600 uA/M/cm and Mean V was expressed in mDO/min. CS activity was measured from a volume of 20 μL (100 μg of total proteins) diluted in 90 μuL of specific reaction buffer (65 μL of 50 mM KH2PO 4 , pH 7.5, 10 μL of Acetyl-CoA 4.4 mM, 10 μL of Oxaloacetate 4.4 mM, 10 μL of 150 mM DTNB). Absorbance at 600 nm was performed over 15 minutes at room temperature (one read per 10 seconds).

### Statistical Analyses

Statistical analyses were performed using Prism (GraphPad). Quantifications were performed blind to genotype.

### Results

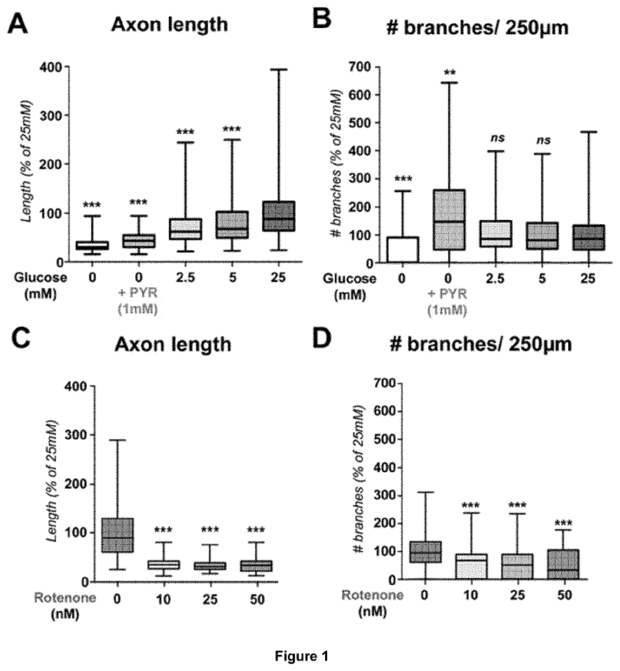

We first sought to identify how a modulation of glucose metabolism in cultured neurons can affect axon development. We observed in primary cultures of cortical neurons a marked dose-response effect of medium glucose concentration on axon development (FIG. 1A-B). Specifically, axonal length increased as a function of glucose concentration. In contrast, even low concentrations of glucose were able to sustain a high number of collateral branches. Even in the absence of glucose, directly fueling the tricarboxylic acid cycle with a low dose of pyruvate (1 mM) was sufficient to support axonal branching. Importantly, high glucose concentration such as the ones classically used for primary cortical and hippocampal neuronal culture favors the production of cellular ATP through glycolysis, whereas mitochondrial metabolism becomes more important in lower glucose concentrations that are closer to physiological conditions (Pathak et al., The Journal of biological chemistry 290, 22325-22336 (2015)). Combined with our results, this demonstrates that culture conditions that favor mitochondrial metabolism are sufficient to support axon branching.

To demonstrate further the importance of axonal mitochondrial metabolism in axonal development, neurons were treated with low doses of rotenone, an inhibitor of OXPHOS complex I, to downregulate mitochondrial oxidative phosphorylation capacity without affecting glycolysis. This led to a decrease of both axon length and collateral branching (FIG. 1C-D). Conversely, we bypassed glycolysis either by replacing glucose by galactose (a 6-carbons sugar catabolized by the glycolytic machinery but negating the net ATP production of glycolysis), or by adding pyruvate to a no-glucose medium. In both case, axon branching was largely equivalent to control conditions (25 mM glucose) despite a strong adverse effect on axon length. Taken together, our results show distinct metabolic requirements for axon elongation and collateral branching and suggest that mitochondrial oxidative metabolism is necessary and sufficient to support axon branching in cortical PNs.

### NUAK1 Kinase Controls Axonal Mitochondrial Metabolism

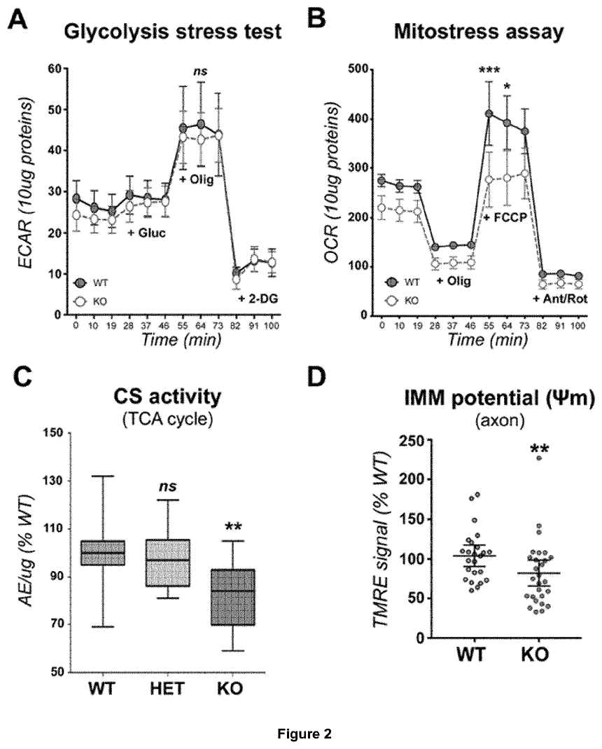

We previously identified that the kinase NUAK1 regulates axon branching in cortical PNs through the control of mitochondria capture at presynaptic boutons (Courchet et al, 2013). In light of this, we aimed at testing a potential role of NUAK1 in controlling mitochondrial metabolism, as recently shown in cancer models (Liu et al. Nature 483, 608-612 (2012), Escalona et al., Frontiers in oncology 10, 1123 (2020)). Through microplate-based respirometry, we measured the glycolytic and respiratory capacity of NUAK1⁺⁺ (WT), NUAK1^{+/-} (HET) or NUAK1^{+/-} neurons (KO). Whereas glycolysis was largely unaffected, our results revealed a trend toward a lower basal respiratory rate, as well as a significant decrease in the maximal respiratory rate upon deletion of NUAK1 (FIG. 2A-B). In parallel, we measured a reduction in the specific activity of the Citrate Synthase (CS), a key enzyme of the TCA cycle, from cortices of NUAK1 KO embryos (FIG. 2C), thus confirming that NUAK1 deletion also alters mitochondrial function in vivo.

The decreased respiratory rate suggests that NUAK1 is required not only for mitochondria trafficking, but also for proper mitochondria function in axons. To test this, we turned to microscopy-based strategies to directly investigate neuronal mitochondrial metabolism with a subcellular resolution. First, we assessed the overall metabolic consequence of NUAK1 knockout with the biosensor PercevalHR. A rapid decrease of PercevalHR signal upon application of Carbonyl cyanide-p-trifluoromethoxyphenylhydrazone (FCCP), a potent uncoupler of mitochondrial oxidative phosphorylations, indicates that most of the ATP: ADP ratio reflects mitochondrial oxidative metabolism. By comparing the basal (FO) and minimum (Fmin, following FCCP application) fluorescence, we observed a significant decrease of PercevalIHR signal in the soma (−21,9%) and in the axon (−23.4%) of NUAK1^{F/F} neurons electroporated with CRE (NUAK1 cKO neurons), indicating a reduction of the ATP: ADP ratio in absence of NUAK1. Next using Tetra-Methyl-Rhodamine Ethyl-ester (TMRE), we measured a marked decrease in mitochondrial inner membrane potential (ΔΨₘ) in the axon of NUAK1 cKO neurons (FIG. 2D). We also observed an increased ROS production by axonal mitochondria in NUAK1 cKO neurons using the mitochondria-targeted biosensor roGFP2. Altogether, our results demonstrate that NUAK1 regulates axonal mitochondrial metabolism, a function that can be uncoupled from its previously demonstrated role in mitochondria trafficking.

### Upregulation of Mitochondria Function Rescues Axon Branching in NUAK1 Deficient Neurons

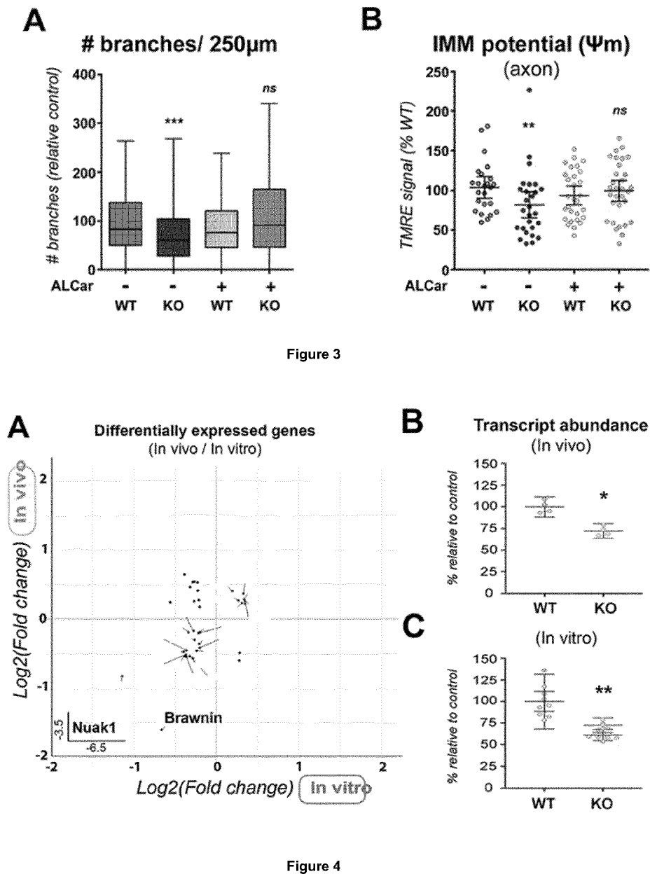

We next devised strategies to demonstrate that impaired axonal metabolic activity is causally linked to the reduction in axon branching observed in NUAK1 mutant neurons. Based on published studies, we treated cortical PNs with the mitochondria modulator acetyl-L-Carnitine (L-Car) (Sainath et al., Developmental neurobiology 77, 454-473 (2017); Dickey et al., The Journal of neuroscience: the official journal of the Society for Neuroscience 31, 15716-15726 (2011)) a cell-permeant, lysine-derived quaternary amine that acts by promoting the metabolism of long-chain fatty acids by the mitochondria. As previously reported (Courchet, (2013)), NUAK1-null neurons had shorter axons with fewer collateral branches. When normalized to axonal length, L-Car induced a complete rescue of collateral branches in NUAK1-deficient neurons (FIG. 3A). As an alternative mean to upregulate mitochondrial function, we used the mitochondria-targeted electron carrier vitamin K2/menaquinone (Vos et al. Science (New York, N.Y.) 336, 1306-1310 (2012)). Similar to the effect of L-Car, we observed a normalization of axonal branching in NUAK1-deficient neurons without affecting axonal length in neurons treated with menaquinone-4 (MK4), an active form of vitamin K2, but not with the inactive form menaquinone-3 (MK3).

We next measured TMRE uptake by mitochondria in 5DIV neuronal cultures. L-Car treatment normalized ΔΨₘ in NUAK1 KO neurons (FIG. 3B), indicating an upregulation of mitochondrial function.

We finally tested if metabolic upregulation can rescue terminal axon branching in vivo by providing L-Car to NUAK1 cKO mice. A conditional inactivation of NUAK1 in the dorsal telencephalon (by breeding with the Nex^{CRE} mouse line (Goebbels et al. (2011)) led to a significant decrease in terminal axon branching in the absence of L-Car supplementation. Strikingly, terminal axon branching was undistinguishable between WT and KO mice when mice were treated with L-Car upon detection of gestation (at E13.5) and during lactation. Taken together, our results demonstrates that a normalization of axonal mitochondria metabolic function is sufficient to rescue axon branching in NUAK1 mutant neurons, demonstrating that more largely, a correction of axonal metabolic deficits can correct abnormal axonal development.

### A Transcriptomic Signature of NUAK1 Deficient Neurons

In order to uncover the mechanism by which NUAK1 regulates axonal mitochondria, we performed bulk RNA sequencing analyses from constitutive NUAK1 KO mice. Whole cortices were collected at E18.5, corresponding to the latest timepoint when we can collect samples from knockout animals who die at birth. Genes over-expressed following NUAK1 KO (i.e. 267 genes) were associated with protein translation, oxidative processes as well as extracellular matrix production and organization. Interestingly, a large fraction of under-expressed genes (i.e. 186 genes), were related to neuron maturation and metabolism, in particular axonogenesis and synaptic transmission. To go further into this analysis, we performed a second round of RNA sequencing from primary neuronal cultures isolated from E15.5 embryos and grown in vitro for 5 days. Since neurons were grown in serum-free medium, there was limited astrocytic growth, allowing to focus our analysis more specifically onto glutamatergic cortical neurons. Genes under-expressed following NUAK1 KO (i.e. 214 genes) were again associated with neuronal maturation, including axogenesis and glutamatergic synapse formation/function, and axonal metabolism.

Genesets Enrichment Analysis (GSEA) highlighted that numerous genesets were similarly perturbed in both conditions (in vivo and in vitro). Among those, genesets related to mitochondria appeared particularly interesting, as they suggest perturbation in mitochondrial function (e.g. ATP production, oxidative phosphorylation).

To identify key genes similarly perturbed by NUAK1 KO and mediating the axonal metabolic imbalance, we performed a new analysis of differentially regulated genes using stringent criteria (Log2FC.threshold>0.25,p<0.01). Intersections of results obtained in vivo and in vitro revealed 37 genes, the majority of which (67%) were affected in similar ways (FIG. 4A). Out of this list we chose to focus on Uqcc6/Brawnin (C12orf73), a small open reading frame (sORF)-encoded peptide (SEP) that was recently identified as an essential factor for mitochondrial supercomplexes assembly (Zhang et al. Nat Commun 11, 1312 (2020); Liang et al. Cell Reports 40, 111204 (2022)).

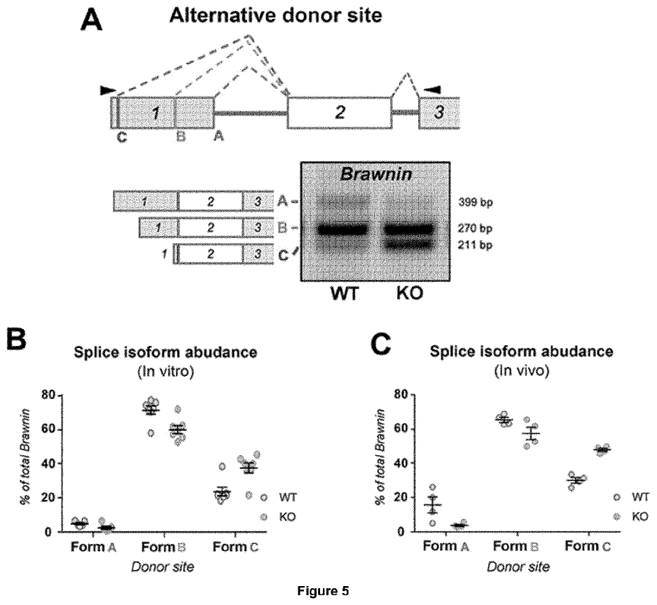

BRAWNIN was ranked 3^{rd} in the list of DEGs in both our in vivo (−67%, pAdj 3.16E-10) and in vitro (−66%, pAdj 4.79E-6) analyses. We could confirm a significant decrease of Brawnin transcript abundance in total mRNA extracts from mouse embryo cortices (−27,6%) (FIG. 4B) as well as from primary neuronal cultures (−38.7%) (FIG. 4C). While analysing the expression pattern of Brawnin by RT-PCR, we observed a distinct pattern of mRNA splicing with the inclusion of alternative donor sites in the non-coding exon 1, producing three alternative splicing isoforms of Brawnin mRNA (FIG. 5A). The longest form, which we termed variant A, includes the whole sequence of exon 1. Two alternative variants included shorter exon 1 and were termed variant B (−129 bp) and variant C (−188 bp). Strikingly, we observed by RT-qPCR a marked decrease of variant A, and to a lesser extent of variant B, and a paralleled increase of the shorter variant C, in both cortices and neurons from Nuak1 KO embryos compared to controls (FIG. 5B-C). Thus, although the coding sequence of Brawnin is not affected by the alternative splicing, our data demonstrate that a loss of Nuak1 affects Brawnin mRNA abundance and splicing, in accordance with the recent observation that NUAK1 regulates the spliceosome complex (Cossa et al. Molecular Cell 77, 1322-1339.e11 (2020)).

### BRAWNIN Expression is Necessary and Sufficient for Cortical Axon Branching

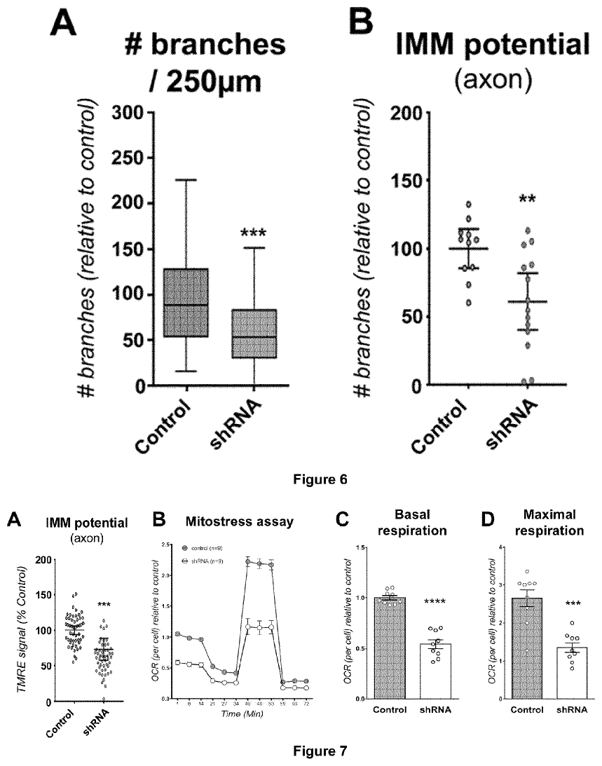

We tested if BRAWNIN might affect axonal metabolism and thus impact axonal development. Inhibition of Brawnin expression with a shRNA led to a marked reduction of collateral branch formation (FIG. 6A). In parallel, the inhibition of Brawnin impaired mitochondria ΔΨₘ, to the same extent as NUAK1 knock out (FIG. 6B). By performing in utero cortical electroporations of two independent shRNA plasmids targeting Brawnin, we demonstrated that BRAWNIN is required for cortical axon development in vivo. Specifically, quantification of axon branching suggested a reduction on the ipsilateral layer V at least upon electroporation of shRNA2 of Brawnin. The reduction of axon branching was more marked on the contralateral side, where either shRNA plasmid targeting Brawnin electroporation led to a marked reduction of terminal branching on both layer II/III and layer V, despite similar fluorescence levels in the WM indicating that axon growth and targeting are not affected. Taken together, our results demonstrate that Brawnin knockdown strongly impacts axonal metabolism and axonal branching.

BRAWNIN inhibition led to a decrease of respiration in cancer cells, but this has never been shown in neurons. To test how BRAWNIN level reduction can decrease neuronal reduction, we knocked down BRAWNIN using shRNA and performed a Seahorse assay to measure mitochondrial respiration. The decrease in BRAWNIN levels resulted in a marked reduction of both basal and maximal respiration (FIG. 7), demonstrating that BRAWNIN is essential to support mitochondrial metabolic activity and collateral branching in developing neurons.

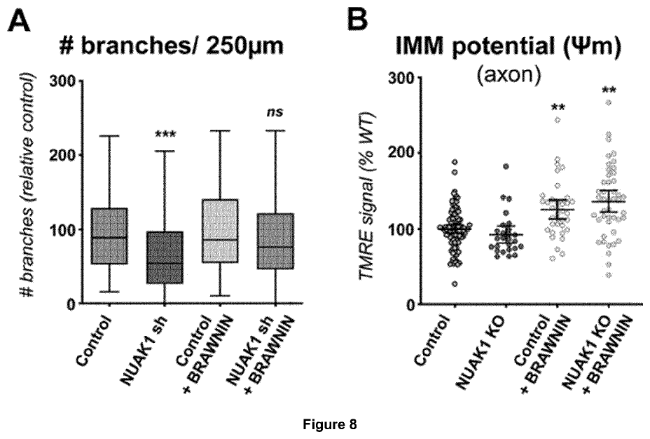

Finally, we tested if BRAWNIN re-expression can correct axonal metabolism deficits and thus rescue axonal development. Using NUAK1 mutant neurons as a model for impaired axonal metabolism, we performed rescue experiments by re-expressing BRAWNIN in Nuak1 knockdown neurons in vitro. As described previously, the inactivation of Nuak1 impaired axon development, leading to shorter axons and fewer collaterals at 5 DIV. Importantly, BRAWNIN completely rescued the branching phenotype induced by Nuak 1-targeting shRNAs (FIG. 8A). To confirm that mitochondrial metabolism is the likely mechanism affected by BRAWNIN to support axon branching, we measured axonal mitochondria ΔΨₘ using TMRE fluorescence. Because this measure is more prone to experimental variability, we used a knockout approach instead of the knockdown strategy. Expression of CRE in Nuak1^{F/F} neurons was accompanied by a reduction in mitochondria ΔΨₘ, which was rescued upon expression of BRAWNIN (FIG. 8B). Overall, our results demonstrate that re-expression of BRAWNIN is sufficient to rescue mitochondria and axon branching phenotypes linked to NUAK1, proving that BRAWNIN is part of a signaling cascade linking NUAK1 to the control of axonal metabolism to support collateral branching in developing axons. Importantly, the overexpression of BRAWNIN also upregulated mitochondrial metabolism in control neurons (FIG. 8B), which strongly suggests that BRAWNIN can correct metabolic activity even when NUAK1 function is not perturbed, and thus that BRAWNIN has a broader therapeutic interest in the treatment of axonal metabolic deficits that extends beyond NUAK1.

### Discussion

Previous studies demonstrated that a deregulation of mitochondria positioning, morphology or function disrupts cortical neurons morphogenesis in vitro (Oruganty-Das, et al. Cell Metabolism 16, 789-800 (2012); Gunnewiek et al. Cell reports 31, 107538 (2020)), as well as the formation of cortical circuits in vivo (Courchet, (2013); Lewis, et al., Nature communications 9, 5008 (2018); Fernandez et al. Neuron 102, 1127-1142.e3 (2019)). In the present study, we provide evidence that mitochondria are preferentially recruited to branch-associated synaptic boutons to support branch stabilization, presumably through metabolic remodeling at the nascent synapse. Furthermore, we describe for the first time that the ASD-linked kinase NUAK1 regulates mitochondrial metabolic activity within cortical neurons axons, through a novel effector, BRAWNIN, whose neuronal functions were previously unknown. Since BRAWNIN is involved in mitochondrial respiratory chain assembly (Zhang (2020); Liang (2022)), our results support a role of BRAWNIN in mediating the effects of NUAK1 on the regulation of mitochondrial activity (Liu (2012); Escalona (2020)).

It is widely accepted that synapses account for the majority of ATP consumption in neurons to ensure ionic homeostasis and synaptic vesicle recycling (Vos et al., Frontiers in Synaptic Neuroscience 2, 139 (2010); Attwell, D. & Laughlin, S. B. Journal of cerebral blood flow and metabolism: official journal of the International Society of Cerebral Blood Flow and Metabolism 21, 1133-1145 (2001); Rangaraju, et al., Cell 156, 825-835 (2014)). In agreement with local metabolic needs, mitochondria are actively transported to and captured at presynapses (Hollenbeck, P. J. & Saxton, W. M. Journal of cell science 118, 5411-5419 (2005); Misgeld, T. & Schwarz, T. L. Neuron 96, 651-666 (2017); Devine, M. J. & Kittler, J. T. Nature Reviews Neuroscience 19, 63-80 (2018)) and support synaptic function through a crosstalk between ATP-producing oxidative phosphorylations (Rangaraju (2014); Ashrafi, et al., Neuron 105, 678-687.e5 (2020)) and calcium buffering (Vaccaro et al., EMBO reports 18, 231-240 (2017); Villegas et al. Journal of Neuroscience 34, 7179-7189 (2014); Kwon et al. PLoS biology 14, e1002516 (2016)).

Our work provides molecular insight into the mechanisms of metabolic regulation in developing neurons, opening perspective for future targeted therapeutics in neurodevelopmental disorders. Especially, mounting evidence supports that altered mitochondrial metabolism is linked to increased risk of various neurodevelopmental disorders including Autism Spectrum Disorders (Chalkia et al. Jama Psychiat 74, 1161(2017); Wang et al.,Plos Genet 12, e1006391 (2016)). Mitochondrial proteins are over-represented in protein interaction network of ASD risk genes (Murtaza et al. Cell Reports 41, 111678 (2022)). Although NUAK1 is classified as a strong candidate (SFARI criteria 2.1) for ASD, our results suggest that the connection to mitochondria metabolism is not through direct protein interaction but rather indirect through the regulation of Brawnin gene expression. Specifically, we observed that NUAK1 depletion alters the differential splicing of Brawnin in its 5′UTR region, in accordance with the description of NUAK1 as a regulator of the spliceosome (Cossa (2020)). Since alternative splicing is accessible to in cellulo and in vivo modulation, our description of the alternative isoforms of Brawnin open avenues for therapies aiming at modulating BRAWNIN expression. Our data demonstrate that the reexpression of BRAWNIN is sufficient to restore mitochondrial function and axon branching in NUAK1 deficient neurons. But more importantly, our data demonstrate that BRAWNIN upregulation is sufficient to increase mitochondrial metabolism in neurons even when NUAK1 expression is normal, indicating that BRAWNIN function is not restricted to NUAK1-induced axonal metabolic disorders but rather that BRAWNIN agonists have a broader therapeutic interest in models of altered mitochondrial function, including neurodevelopment (e.g. autism spectrum disorders) and neurodegeneration (e.g. mitochondrial ataxia, SMA or CMT).

1-9. (canceled)

10. A method for screening candidate therapeutic compounds, wherein said method comprises:
a. Culturing a cellular model of neural cells in presence of a candidate compound;
b. Assaying the effects of said candidate compound on the expression level of Brawnin in said cellular model; and
c. Selecting said compound based on said effects.

11. The method according to claim 10, wherein said cellular model is a model of an axonal metabolic disorder.

12. A method for the treatment of axonal metabolic disorders in a patient in need thereof, said method comprising administering a therapeutically effective amount of a BRAWNIN agonist to said patient.

13. The method according to claim 1, wherein said axonal metabolic disorder is an Autism Spectrum Disorder (ASD).

14. The method according to claim 1, wherein said axonal metabolic disorder is Charcot-Marie-Tooth disease.

15. The method according to claim 1, wherein said axonal metabolic disorder is a mitochondrial ataxia.

16. The method according to claim 1, wherein said axonal metabolic disorder is Spinal Muscular Atrophy (SMA).

17. The method according to claim 1, wherein said BRAWNIN agonist is the BRAWNIN protein or a functional fragment thereof.

18. The method according to claim 1, wherein said BRAWNIN agonist is a BRAWNIN-encoding polynucleotide.

19. The method according to claim 1, wherein said BRAWNIN agonist is a nucleotide sequence allowing for expression of the BRAWNIN gene.

20. The method according to claim 19, wherein said nucleotide sequence allowing for the expression of the BRAWNIN gene is an antisense RNA molecule or a small interfering RNA (siRNA).
